(19) United States
(12) Patent Application Publication
(10) Pub. No.: US 2024/0138709 A1
(43) Pub. Date: May 2, 2024

(54) CARE SYSTEM MONITORING METHOD AND CARE SYSTEM

(71) Applicant: InnoLux Corporation, Miao-Li County (TW)

(72) Inventors: Cheng-Hsu CHOU, Miao-Li County (TW); Wei-Chih LIU, Miao-Li County (TW); Fang-Iy WU, Miao-Li County (TW)

(21) Appl. No.: 18/462,521

(22) Filed: Sep. 7, 2023

(30) Foreign Application Priority Data
Oct. 14, 2022 (CN) 202211261051.0

(51) Int. Cl. A61B 5/11; A61B 5/00

(52) U.S. Cl. CPC A61B 5/1114; A61B 5/746; A61B 5/6892; A61B 2562/0247

(57) ABSTRACT

A care system monitoring method for sensing the movement state of a bedridden person is provided. The care system monitoring method includes the following stages. Initial-state information is sensed. A first warning signal is issued to remind the caregiver to perform a movement action on the bedridden person when the time that the bedridden person has been in the initial state exceeds the threshold period. First-state information (which is information that is collected when the bedridden person is in the first state) is sensed after completing the movement action. It is determined whether the bedridden person was moved correctly according to the initial-state information and the first-state information. The first-state information is reset as the initial-state information if it is determined that the bedridden person has been moved correctly. A second warning signal is issued if it is determined that the bedridden person has been moved incorrectly.

### CROSS REFERENCE TO RELATED APPLICATIONS

This application claims the benefit of China Application No. 202211261051.0, filed on Oct. 14, 2022, the entirety of which is incorporated by reference herein.

### BACKGROUND OF THE DISCLOSURE

### Field of the Invention

The present invention relates to a monitoring method, and, in particular, to a care system monitoring method and a care system.

### Description of the Related Art

When a patient (such as an elderly person, one with limited mobility, or an injured or sick person) is bedridden and forced to sit or lie in bed for extended periods of time—often because they cannot turn or move independently—the cells and tissues are under pressure for a long time, resulting in blood flow obstructions, necrosis, and the formation of decubitus sores (bedsores). Even if there are caregivers to regularly help turn and change the body position, decubitus sores may occur due to insufficient body position changes. Therefore, how to assist in regular reminders to turn the patient and judge whether the body position change is sufficient has become an important issue.

### BRIEF SUMMARY OF THE DISCLOSURE

An embodiment of the present disclosure provides a care system monitoring method for sensing the movement state of a bedridden person. The care system monitoring method includes the following states. Initial-state information (which is information that is collected when the bedridden person is in the initial state) is sensed. A first warning signal is issued to remind the caregiver to perform a movement action on the bedridden person when the time that the bedridden person has been in the initial state exceeds the threshold period. First-state information (which is information that is collected when the bedridden person is in the first state) is sensed after completing the movement action. It is determined whether the bedridden person was moved correctly according to the initial-state information and the first-state information. The first-state information is reset as the initial-state information if it is determined that the bedridden person has been moved correctly. A second warning signal is issued if it is determined that the bedridden person has been moved incorrectly.

An embodiment of the present disclosure also provides a care system. The care system includes a first sensor, a signal analysis unit, and a warning unit. The first sensor senses initial-state information before a movement action is performed on the bedridden person and senses first-state information after the movement action has been performed on the bedridden person. The signal analysis unit receives the initial-state information, determines whether the time that the bedridden person has been in an initial state exceeds the threshold period, and issues a first warning signal if the time that the bedridden person has been in the initial state exceeds the threshold period. The warning unit receives the first warning signal and notifies the caregiver to perform the movement action on the bedridden person. The signal analysis unit further receives the first-state information, and determines whether the bedridden person was moved correctly according to the initial-state information and the first-state information after the movement action has been performed on the bedridden person. The signal analysis unit resets the first-state information as the initial-state information if it is determined that the bedridden person has been moved correctly. The signal analysis unit issues a second warning signal if it is determined that the bedridden person has been moved incorrectly.

### BRIEF DESCRIPTION OF THE DRAWINGS

The disclosure can be more fully understood by reading the subsequent detailed description with references made to the accompanying figures. It should be understood that the figures are not drawn to scale in accordance with standard practice in the industry. In fact, it is allowed to arbitrarily enlarge or reduce the size of components for clear illustration. This means that many special details, relationships and methods are disclosed to provide a complete understanding of the disclosure.

FIGS. 1A and 1B is a flow chart of a care system monitoring method in accordance with some embodiments of the present disclosure.

FIG. 2A is a schematic diagram of a care system 200 in accordance with some embodiments of the present disclosure.

FIG. 2B is a schematic diagram of a care system 220 in accordance with some embodiments of the present disclosure.

FIG. 3 is a schematic diagram of a signal analysis unit 208 in accordance with some embodiments of the present disclosure.

FIG. 4A is a schematic diagram of sensing a movement sate of a bedridden person by using a first sensor in accordance with some embodiments of the present disclosure.

FIG. 4B is a schematic diagram of sensing the movement sate of the bedridden person by using a second sensor in accordance with some embodiments of the present disclosure.

FIG. 4C is a schematic diagram of sensing the movement sate of the bedridden person by using the first sensor and the second sensor in accordance with some embodiments of the present disclosure.

FIG. 5A is a schematic diagram of sensing by using the first sensor and slapping the back of the bedridden person in accordance with some embodiments of the present disclosure.

FIG. 5B is a schematic diagram of sensing by using the first sensor and slapping the back of the bedridden person in accordance with some embodiments of the present disclosure.

### DETAILED DESCRIPTION OF THE DISCLOSURE

In order to make the above purposes, features, and advantages of some embodiments of the present disclosure more comprehensible, the following is a detailed description in conjunction with the accompanying drawing.

Certain terms are used throughout the description and following claims to refer to particular components. As one skilled in the art will understand, electronic equipment manufacturers may refer to a component by different names. This document does not intend to distinguish between components that differ in name but not function. It is understood that the words “comprise”, “have” and “include” are used in an open-ended fashion, and thus should be interpreted to mean “include, but not limited to . . . ”. Thus, when the terms “comprise”, “have” and/or “include” used in the present disclosure are used to indicate the existence of specific technical features, values, method steps, operations, units and/or components. However, it does not exclude the possibility that more technical features, numerical values, method steps, work processes, units, components, or any combination of the above can be added.

The directional terms used throughout the description and following claims, such as: “on”, “up”, “above”, “down”, “below”, “front”, “rear”, “back”, “left”, “right”, etc., are only directions referring to the drawings. Therefore, the directional terms are used for explaining and not used for limiting the present disclosure. Regarding the drawings, the drawings show the general characteristics of methods, structures, and/or materials used in specific embodiments. However, the drawings should not be construed as defining or limiting the scope or properties encompassed by these embodiments. For example, for clarity, the relative size, thickness, and position of each layer, each area, and/or each structure may be reduced or enlarged.

When the corresponding component such as layer or area is referred to as being “on another component”, it may be directly on this other component, or other components may exist between them. On the other hand, when the component is referred to as being “directly on another component (or the variant thereof)”, there is no component between them. Furthermore, when the corresponding component is referred to as being “on another component”, the corresponding component and the other component have a disposition relationship along a top-view/vertical direction, the corresponding component may be below or above the other component, and the disposition relationship along the top-view/vertical direction is determined by the orientation of the device.

It should be understood that when a component or layer is referred to as being “connected to” another component or layer, it can be directly connected to this other component or layer, or intervening components or layers may be present. In contrast, when a component is referred to as being “directly connected to” another component or layer, there are no intervening components or layers present.

The electrical connection or coupling described in this disclosure may refer to direct connection or indirect connection. In the case of direct connection, the endpoints of the components on the two circuits are directly connected or connected to each other by a conductor line segment, while in the case of indirect connection, there are switches, diodes, capacitors, inductors, resistors, other suitable components, or a combination of the above components between the endpoints of the components on the two circuits, but the intermediate component is not limited thereto.

The words “first”, “second”, “third”, “fourth”, “fifth”, and “sixth” are used to describe components. They are not used to indicate the priority order of or advance relationship, but only to distinguish components with the same name.

It should be noted that the technical features in different embodiments described in the following can be replaced, recombined, or mixed with one another to constitute another embodiment without departing from the spirit of the present disclosure.

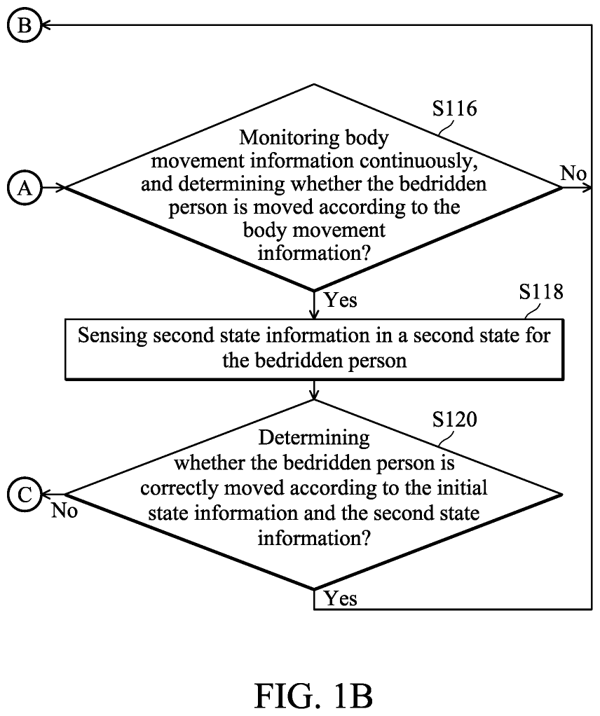

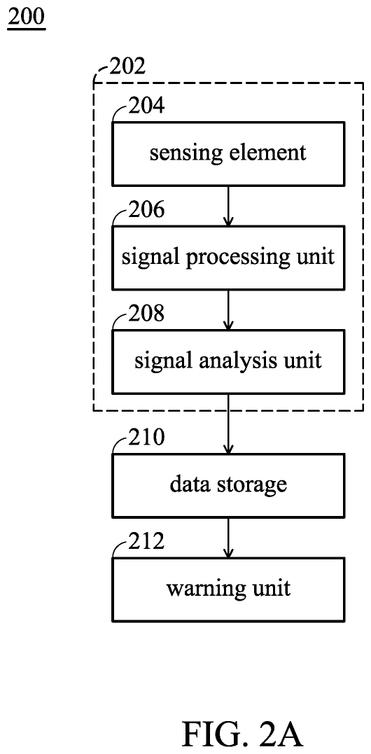

In the present disclosure, a care system in FIG. 2A and a care system in FIG. 2B in the present disclosure may include at least one electronic device. The electronic device may include a display device, a backlight device, an antenna device, a sensing device, or a splicing device, etc., but is not limited thereto. The electronic device may be a bendable or flexible electronic device. The display device may be a non-self-luminous display device or a self-luminous display device. The antenna device may be a liquid crystal antenna device or a non-liquid crystal antenna device, and the sensing device may be a sensing device for sensing capacitance, light, heat, or ultrasonic waves, but is not limited thereto. The electronic components may include passive and active components, such as capacitors, resistors, inductors, diodes, transistors, and the like. The diodes may include light-emitting diodes or photodiodes. The light-emitting diode may include organic light-emitting diode (OLED), inorganic light-emitting diode, micro-LED, mini-LED, quantum dot light-emitting diode (QLED, QDLED), other suitable materials or a combination of the above materials, but is not limited thereto. The splicing device may be, for example, a splicing display device or a splicing antenna device, but is not limited thereto. In addition, the display device in the electronic device may be a color display device or a monochrome display device, and the shape of the electronic device may be rectangular, circular, polygonal, a shape with curved edges, or other suitable shapes. In addition, the electronic device described below uses, as an example, the sensing of a touch through an embedded touch device, but the touch-sensing method is not limited thereto, and another suitable touch-sensing method can be used provided that it meets all requirements.

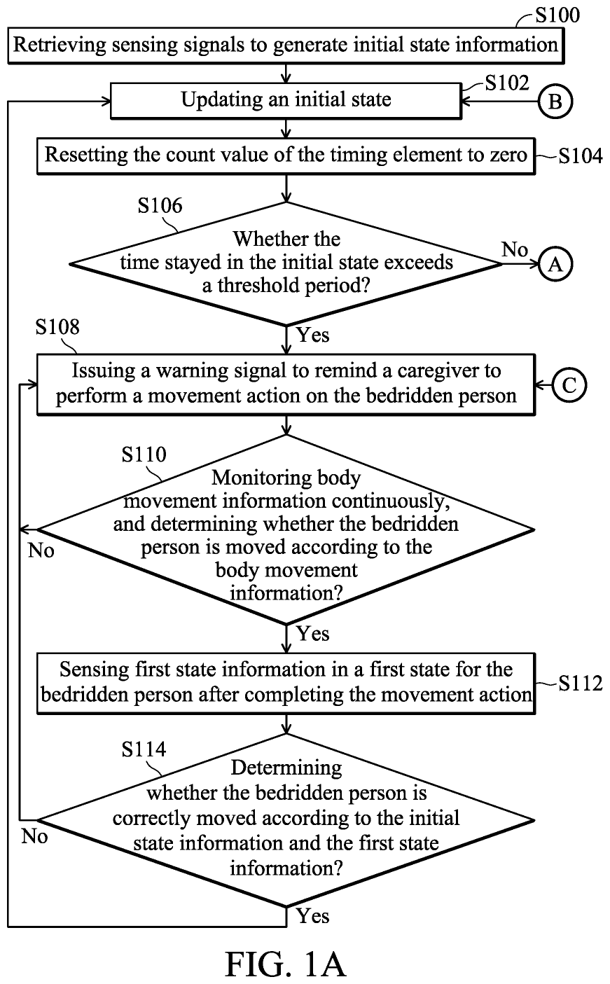

FIGS. 1A and 1B are a flow chart of a care system monitoring method in accordance with some embodiments of the present disclosure. As shown in FIGS. 1A and 1B, the care system monitoring method of the present disclosure includes the following stages. Initial-state information (which is information that is collected when the bedridden person is in the initial state) is sensed (for example, step S100 plus step S102). The count value of the timing element is reset to zero (for example, step S104). When the time that the bedridden person has been in the initial state exceeds the threshold period (for example, “Yes” in step S106), a first warning signal is issued to remind the caregiver to perform a movement action on the bedridden person (for example, step S108). When the time that the bedridden person has been in the initial state exceeds the threshold period, body-movement information is monitored continuously and it determined whether the bedridden person is moved according to the body-movement information (for example, step S110). First-state information is sensed after completing the movement action (for example, step S112). It is determined whether the bedridden person was moved correctly according to the initial-state information and the first-state information (for example, step S114). In some embodiments, if it is determined that the bedridden person has been moved correctly (for example, “Yes” in step S114), the initial-state information is reset to the first-state information (for example, the method returns to step S102). If it is determined that the bedridden person has been moved incorrectly (for example, “No” in step S114), a second warning signal is issued (for example, the method returns to step S108).

The care system monitoring method of the present disclosure further includes the following stages. When the time that the bedridden person has been in the initial state does not exceed the threshold period (for example, “No” in step S106), body-movement information is monitored continuously and it determined whether the bedridden person is moved according to the body-movement information (for example, step S116). Second-state information in a second state is sensed after the bedridden person has been moved, and it is determined whether the bedridden person was moved correctly according to the initial-state information and the second-state information (step S120). In some embodiments, if it is determined that the bedridden person has been moved correctly (for example, “Yes” in step S120), the second-state information is reset as the initial-state information (for example, the method returns to step S102). If it is determined that the bedridden person has been moved incorrectly (for example, “No” in step S120), a third warning signal is issued (for example, the method returns to step S108).

In detail, in step S100, the care system monitoring method of the present disclosure senses the sensing signal from the body of the bedridden person through a sensor when the body of the bedridden person is on the bed. In some embodiments, the sensor may be, for example, a pressure sensor, an image sensor, an infrared light sensor, or a combination of the above sensors, but the present disclosure is not limited thereto. The sensor may include, for example, at least two sensing elements corresponding to different body parts of the bedridden person, and may output sensing signals corresponding to different body parts through the sensing elements, and correspondingly generate initial-state information. For example, when the sensor is a pressure sensor, the sensing element may output, for example, the sensed pressure value corresponding to the body part as the sensing signal. In some embodiments, the sensor is an image sensor, for example, the concept of coordinates can be used to output the sensing position of the corresponding body part. For example, the middle of the bed is the zero point, the right side of the bed is the positive direction, and the left side of the bed is the negative direction, but the present disclosure is not limited thereto. For example, when the bedridden person is lying supine, the coordinates of the sensing element corresponding to the right side of the bedridden person's body are located on the right side of the bed, and the sensing value of the sensing element on the right side is positive. In contrast, the coordinates of the sensing element corresponding to the left side of the bedridden person's body are located on the left side of the bed, the sensing value of the sensing element on the left side is negative. For another example, when the bedridden person is lying in the right lateral position, the coordinates of the sensing element corresponding to the right side of the bedridden person's body and the coordinates of the sensing element corresponding to the left side of the bedridden person's body are located on the right side of the bed. Therefore, the sensing values of the sensing element corresponding to the right side of the bedridden person's body and the sensing element corresponding to the left side of the bedridden person's body are both positive, but the present disclosure is not limited thereto. The care system monitoring method of the present disclosure generates initial-state information correspondingly according to the sensing signals from the sensors. The care system monitoring method of the present disclosure performs analog-to-digital conversion, filtering, and amplification on the sensing signals from the sensing elements to generate initial-state information. In some embodiments, the care system may have many different types of sensors, for example. Through the combined application of different sensors, it is possible to more accurately determine the movement state of the bedridden person on the bed, or to further analyze different detailed information. For example, for the bedridden person who cannot bear too much pressure on a specific part, the movement of the bedridden person on the bed can be sensed through the image sensor, and the pressure sensor can be used to help understand the pressure value of the bedridden person every time the specific part moves. In this way, it is possible to track whether the specific part is under excessive pressure and cause discomfort, analyze and adjust the movement method according to the needs of different bedridden persons, so as to achieve the best user experience, but the present disclosure is not limited thereto.

In step S102, the care system monitoring method of the present disclosure determines that the bedridden person is in an initial state according to the initial-state information. In some embodiments, the initial state may be, for example, the state of lying supine, the state of lying on the ride side, or the state of lying in the left lateral position, but the present disclosure is not limited thereto. For example, the care system monitoring method of the present disclosure converts the respective output sensing signals of the sensing elements in the sensor into corresponding initial-state information (such as posture state judgment parameters). The care system monitoring method of the present disclosure determine whether the bedridden person is lying supine or lying in the right lateral position, or lying in the left lateral position according to the relationship between the posture state judgment parameters corresponding to each sensing element. For example, the relationship between the posture state judgment parameters may be, for example, the sum of the differences of the posture state judgment parameters, the quotient of the posture state judgment parameters, and the setting threshold. In some embodiments, the setting threshold is related to the type of sensor, the size of the sensor, and the body shape and weight of the bedridden person, but the present disclosure is not limited thereto. In step S104, the care system monitoring method of the present disclosure resets the count value of the timing element to zero to start counting the time when the bedridden person is in the initial state. In step S106, if the time that the bedridden person has been in the initial state exceeds the threshold period (for example, 2 hours), the care system monitoring method of the present disclosure performs step S108. If the time that the bedridden person has been in the initial state does not exceed the threshold period, the care system monitoring method of the present disclosure then performs step S116.

In step S108, the care system monitoring method of the present disclosure issues a warning signal to remind the caregiver to perform a movement action on the bedridden person. In some embodiments, the movement action in step S108 is turning, for example, but the present disclosure is not limited thereto. In some embodiments, after the warning signal is issued, if the bedridden person fails to be turned over within a preset time, the care system monitoring method of the present disclosure may directly notify the care station or family members, but the present disclosure is not limited thereto. In step S110, the care system monitoring method of the present disclosure continuously monitors the body-movement information, and determines whether the bedridden person is moved according to the body-movement information. In detail, the care system monitoring method of the present disclosure determines whether the bedridden person is moved according to whether the signal amplitude of the initial-state information corresponding to each sensing element is larger than an amplitude threshold. For example, when the signal amplitude of the initial-state information corresponding to the plurality of sensing elements is larger than the amplitude threshold, the care system monitoring method of the present disclosure determines that the bedridden person is moved. If the bedridden person is moved (“Yes” in step S110), the care system monitoring method of the present disclosure senses another set of state information after the movement (that is, step S112), and determines whether the bedridden person has been moved correctly through the difference of the state information before and after (that is, step S114). In contrast, when the signal amplitude of the initial-state information corresponding to the sensing elements are less than or equal to the amplitude threshold, the care system monitoring method of the present disclosure determines that the bedridden person is not moved. If the bedridden person has not been moved (“No” in step S110), the care system monitoring method of the present disclosure does not need to record or determine any information, and the method returns to step S108.

In step S114, the care system monitoring method of the present disclosure determines whether the bedridden person has been moved correctly according to the relationship between the posture state judgment parameters corresponding to each sensing element. For example, the relationship between the parameters may be, for example, the sum of the differences of the posture state judgment parameters, the quotient of the posture state judgment parameters, and the setting threshold. In some embodiments, the setting threshold is related to the type of sensor, the size of the sensor, and the body shape and weight of the bedridden person, but the present disclosure is not limited thereto. In some embodiments, the care system monitoring method of the present disclosure may perform automatic calibration on the setting threshold when the first care monitoring is performed. In some embodiments, if it is determined that the bedridden person has been moved correctly (“Yes” in step S114), before step S102 is performed, the care system monitoring method of the present disclosure activates the device for relieving stress concentration, so that the bedridden person can have a better user experience. In some embodiments, the device for relieving stress concentration can be, for example, an electric mattress, which can perform pressure relieving actions on the body parts of the bedridden person according to settings, but the present disclosure is not limited thereto. After it is determined that the bedridden person has been moved correctly, the care system monitoring method of the present disclosure resets the first-state information sensed in step S112 as the initial-state information: That is, the initial state of the bedridden person is updated (for example, step S102). If it is determined that the bedridden person has been moved incorrectly (“No” in step S114), the care system monitoring method of the present disclosure returns to step S108. In some embodiments, the care system monitoring method of the present disclosure further monitors the first-state information of the bedridden person after the movement action, determines whether the caregiver slaps the bedridden person's body, so as to increase the functionality of the care system monitoring method disclosed in the present disclosure. In some embodiments, step S114 further includes the determination of the repetition (posture) state. For example, the care system monitoring method of the present disclosure records and compares the previous two (posture) states of the bedridden person, and the previous two (posture) states of the bedridden person cannot be repeated. In some embodiments, the care system monitoring method of the present disclosure records and compares the previous one state of the bedridden person, and the previous one (posture) state of the bedridden person cannot be repeated.

In other words, when the previous two or one (posture) state of the bedridden person is not repeated, the care system monitoring method of the present disclosure determines that step S114 is “Yes”. If it is determined that the bedridden person has been moved correctly (that is, “Yes” in step S114), the care system monitoring method of the present disclosure resets the first-state information as the initial-state information (that is, the method returns to step S102). If it is determined that the bedridden person has been moved incorrectly (that is, “No” in step S114), the care system monitoring method of the present disclosure issues the warning signal (that is, the method returns to step S108). In some embodiments, the care system monitoring method of the present disclosure further includes the warning signal for power outage and signal interruption, but the present disclosure is not limited thereto.

When the time that the bedridden person has been in the initial state does not exceeds the threshold period (“No” in step S106), the care system monitoring method of the present disclosure continuously performs step S116. In step S116, the care system monitoring method of the present disclosure continuously monitors the body-movement information, and determines whether the bedridden person is moved according to the body-movement information. In detail, the care system monitoring method of the present disclosure determines whether the bedridden person is moved according to whether the signal amplitude of the initial-state information corresponding to each sensing element is larger than the amplitude threshold (the same as step S112). If it is determined that the bedridden person has been moved (“Yes” in step S116), the care system monitoring method of the present disclosure continuously performs step S118. If the bedridden person is determined to be not moved (“No” in step S116), the care system monitoring method of the present disclosure returns to step S106, and performs subsequent steps according to whether the time that the bedridden person has been in the initial state exceeds the threshold period. In step S118, the care system monitoring method of the present disclosure sense the second-state information in the second state for the bedridden person.

Afterwards, in step S120, the care system monitoring method of the present disclosure determines whether the bedridden person has been moved correctly according to the initial-state information in step S100 and the second-state information in step S118. In some embodiments, the care system monitoring method of the present disclosure determines whether the bedridden person has been moved correctly according to the relationship between the posture state judgment parameters corresponding to each sensing element. For example, the relationship between the parameters may be, for example, the sum of the differences of the posture state judgment parameters, the quotient of the posture state judgment parameters, and the setting threshold. In some embodiments, the setting threshold is related to the type of sensor, the size of the sensor, and the body shape and weight of the bedridden person, but the present disclosure is not limited thereto. In some embodiments, the care system monitoring method of the present disclosure may perform automatic calibration on the setting threshold when the first care monitoring is performed. In some embodiments, step S120 further includes the determination of the repetition (posture) state. For example, the care system monitoring method of the present disclosure records and compares the previous two (posture) states of the bedridden person, and the previous two (posture) states of the bedridden person cannot be repeated. In some embodiments, the care system monitoring method of the present disclosure records and compares the previous one state of the bedridden person, and the previous one (posture) state of the bedridden person cannot be repeated.

In other words, when the previous two or one (posture) state of the bedridden person is not repeated, the care system monitoring method of the present disclosure determines that step S120 is “Yes”. If it is determined that the bedridden person has been moved correctly (that is, “Yes” in step S114), the care system monitoring method of the present disclosure resets the second-state information as the initial-state information (that is, the method returns to step S102). If it is determined that the bedridden person has been moved incorrectly (that is, “No” in step S120), the care system monitoring method of the present disclosure issues the warning signal (that is, the method returns to step S108). In some embodiments, the care system monitoring method of the present disclosure further includes the warning signal for power outage and signal interruption, but the present disclosure is not limited thereto.

FIG. 2A is a schematic diagram of a care system 200 in accordance with some embodiments of the present disclosure. As shown in FIG. 2A, the care system 200 includes a sensor 202, a data storage 210, and a warning unit 212. In some embodiments, the sensor 202 includes a sensing element 204, a signal processing unit 206, and a signal analysis unit 208. In some embodiments, the sensor 202 may be, for example, a pressure sensor, an image sensor, an infrared light sensor, or a combination of the above sensors, but the present disclosure is not limited thereto. If the sensor 202 is a pressure sensor, the sensing element 204 may be, for example, disposed on the bed corresponding to different body parts of the bedridden person or on the clothes of the bedridden person, but the present disclosure is not limited thereto. If the sensor 202 is an image sensor, the sensing element 204 can be, for example, a photosensitive element in a lens, but the present disclosure is not limited thereto. In some embodiments, the sensor 202 senses initial-state information before the movement action performed on the bedridden person (step S100, step S102 in FIG. 1A) and first-state information after the movement action performed on the bedridden person (step S112 in FIG. 1A). The signal processing unit 206 performs analog-to-digital conversion, filtering, and amplification on the sensing signal from the sensing element 204, so as to generates the initial-state information and the first-state information after the movement action. The signal analysis unit 208 receives the initial-state information and determines whether the time that the bedridden person has been in the initial state exceeds the threshold period (step S106 in FIG. 1A). If the time that the bedridden person has been in the initial state exceeds the threshold period (“Yes” in step S106 in FIG. 1A), a warning signal is output (step S108 in FIG. 1A).

In some embodiments, when the signal analysis unit 208 detects that the time that the bedridden person has been in the initial sate exceeds the threshold period, the signal analysis unit 208 continuously monitors and receives body-movement information, and determines whether the bedridden person is moved according to the body-movement information (step S110 in FIG. 1A). In some embodiments, when the signal analysis unit 208 determines that the bedridden person is moved (“Yes” in step S110 in FIG. 1A), the signal analysis unit 208 further receives the first-state information (step S112 in FIG. 1A). The signal analysis unit 208 determines whether the bedridden person has been moved correctly according to the initial-state information and the first-state information after the movement action has been performed on the bedridden person (step S114 in FIG. 1A). The signal analysis unit 208 determines whether the bedridden person has been moved correctly according to the relationship between the posture state judgment parameters corresponding to each sensing element 204. For example, the relationship between the parameters may be, for example, the sum of the differences of the posture state judgment parameters, the quotient of the posture state judgment parameters, and the setting threshold. If it is determined that the bedridden person has been moved correctly (“Yes” in step S114 in FIG. 1A), the signal analysis unit 208 resets the first-state information as the initial-state information (step S102 in FIG. 1A). If it is determined that the bedridden person has been moved incorrectly (“No” in step S114 in FIG. 1A), the signal analysis unit 208 outputs a warning signal (step S108 in FIG. 1). In some embodiments, when the signal analysis unit 208 determines that the bedridden person has not been moved (“No” in step S110 in FIG. 1A), the signal analysis unit 208 outputs the warning signal again (step S108 in FIG. 1A). In some embodiments, the warning unit 212 receives the warning signal, and notifies the caregiver to perform the movement action on the bedridden person. The warning unit 212 may be, for example, a personal computer of a monitoring center or a mobile device of a medical caregiver, but the present disclosure is not limited thereto.

In some embodiments, if the time that the bedridden person has been in the initial state does not exceed the threshold period (“No” in step S106 in FIG. 1A), the signal analysis unit 208 continuously monitors and receives body-movement information, and determines whether the bedridden person is moved according to the body-movement information (step S116 in FIG. 1B). When the signal analysis unit 208 determines that the bedridden person is moved (“Yes” in step S116 in FIG. 1B), the signal analysis unit 208 senses second-state information in a second state after the movement action has been performed on the bedridden person (step S118 in FIG. 1B). The signal analysis unit 208 determines that the bedridden person has been moved correctly according to the initial-state information and the second-state information (step S120 in FIG. 1B). The signal analysis unit 208 determines whether the bedridden person has been moved correctly according to the relationship between the posture state judgment parameters corresponding to each sensing element 204. For example, the relationship between the parameters may be, for example, the sum of the differences of the posture state judgment parameters, the quotient of the posture state judgment parameters, and the setting threshold. When the signal analysis unit 208 determines that the bedridden person has been moved correctly (“Yes” in step S120 in FIG. 1), the signal analysis unit 208 resets the second-state information as the initial-state information (step S102 in FIG. 1A). When the signal analysis unit 208 determines that the bedridden person has been moved incorrectly (“No” in step S120 in FIG. 1), the signal analysis unit 208 issues a warning signal (step S108 in FIG. 1A).

In some embodiments, the signal analysis unit 208 further performs the determination on the repetition (posture) state. In other words, when the previous two or one (posture) state of the bedridden person is not repeated, the signal analysis unit 208 determines that the bedridden person is moved correctly. In some embodiments, when the signal analysis unit 208 determines that the bedridden person has not been moved (“No” in step S116 in FIG. 1B), the signal analysis unit 208 determines whether the time that the bedridden person has been in the initial state exceeds the threshold period again (step S106 in FIG. 1A). In some embodiments, the data storage 210 receives and stores the initial-state information, the first-state information, and the second-state information.

FIG. 2B is a schematic diagram of a care system 220 in accordance with some embodiments of the present disclosure. As shown in FIG. 2B, the care system 220 includes a sensor 222, a data storage 224, and a warning unit 212. In some embodiments, the sensor 222 includes the sensing element 204 and the signal processing unit 206. The data storage 224 includes the signal analysis unit 208. The biggest difference between the care system 220 and the care system 200 is that in the care system 220, the signal analysis unit 208 is disposed in the data storage 224 (such as a cloud server). In the care system 200, the signal analysis unit 208 is disposed in the sensor 202 (such as a pressure sensor or an image sensor). The actions performed by other components in the care system 220 are the same as those performed by other components in the care system 200, so they will not be repeated here. In some embodiments, the signal analysis unit can be disposed in the sensor and the data storage at the same time (not shown). For example, the first signal analysis unit (not shown) in the sensor can perform the determination of the state (posture) of the bedridden person, and the second signal analysis unit (not shown) in the data storage can determine whether the bedridden person is moved correctly, but the present disclosure is not limited thereto.

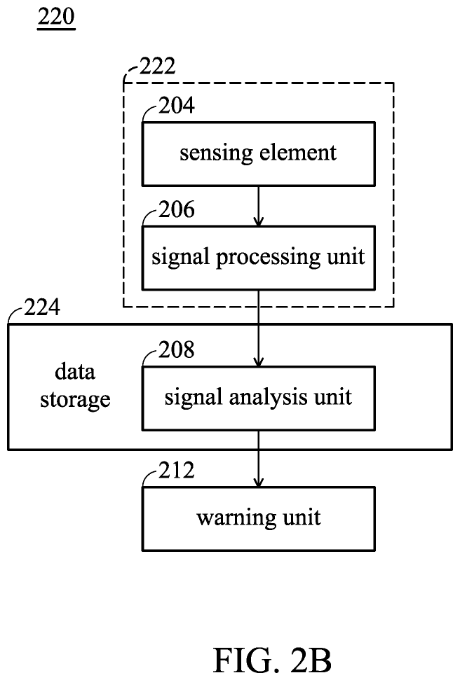

FIG. 3 is a schematic diagram of a signal analysis unit 208 in accordance with some embodiments of the present disclosure. As shown in FIG. 3, the signal analysis unit 208 includes, for example, a timing element 300 and/or an analysis element 302. The timing element 300 determines whether the time that the bedridden person has been in the initial state exceeds the threshold period. If the time that the bedridden person has been in the initial state exceeds the threshold period, the timing element 300 outputs the warning signal. In some embodiments, the timing element 300 may be, for example, a counter, but the present disclosure is not limited thereto. The analysis element 302 determines whether the bedridden person has been moved correctly according to the initial-state information and the other state information. In some embodiments, the analysis element 302 may be a microprocessor that executes (posture) status determination and correct movement determination, but the present disclosure is not limited thereto.

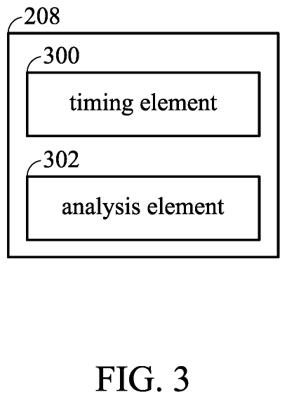

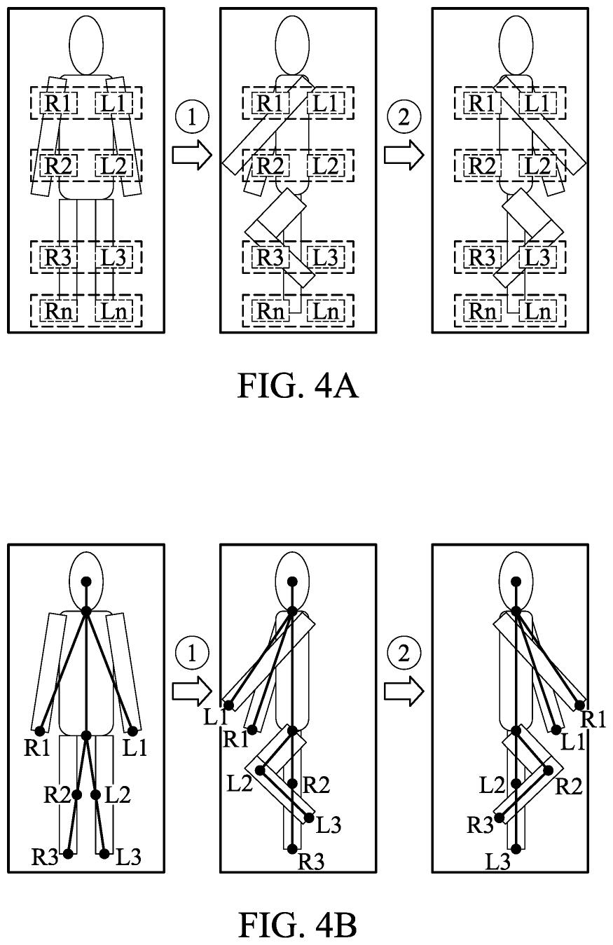

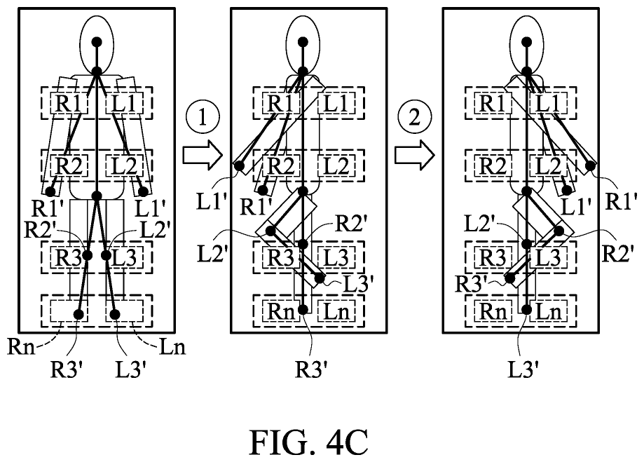

FIG. 4A is a schematic diagram of sensing a movement sate of a bedridden person by using a first sensor in accordance with some embodiments of the present disclosure. In some embodiments, the first sensor is a pressure sensor. The first sensor includes at least two sensing elements corresponding to different body parts of the bedridden person. For example, The first sensor of FIG. 4A includes a first right sensing element, a second right sensing element, a third right sensing element, . . . , a nth right sensing element, a first left sensing element, a second left sensing element, a third left sensing element, . . . , and a nth left sensing element. Wherein n is a positive integer. In some embodiments, in cases where the initial state of the bedridden person is lying supine, the first right sensing element is arranged on the bed under the right arm of the bedridden person. The second right sensing element is arranged on the bed below the right elbow of the bedridden person. The third right sensing element is arranged on the bed below the right knee of the bedridden person. The nth right sensing element is arranged on the bed below the right footboard of the bedridden person. Similarly, in cases where the initial state of the bedridden person is lying supine, the first left sensing element is arranged on the bed under the left arm of the bedridden person. The second left sensing element is arranged on the bed below the left elbow of the bedridden person. The third left sensing element is arranged on the bed below the left knee of the bedridden person. The nth left sensing element is arranged on the bed below the left footboard of the bedridden person.

The signal analysis unit 208 converts the respective output sensing signals of each sensing element 204 into corresponding state information, and generates corresponding posture state judgment parameters. For example, the signal analysis unit 208 may obtain the posture state judgment parameter R1 corresponding to the first right sensing element, the posture state judgment parameter R2 corresponding to the second right sensing element, the posture state judgment parameter R3 corresponding to the third right sensing element, and the posture state judgment parameter Rn corresponding to the nth right sensing element. The signal analysis unit 208 may also obtain the posture state judgment parameter L1 corresponding to the first left sensing element, the posture state judgment parameter L2 corresponding to the second left sensing element, the posture state judgment parameter L3 corresponding to the third left sensing element, and the posture state judgment parameter Ln corresponding to the nth left sensing element.

The signal analysis unit 208 determines whether the bedridden person has been moved correctly according to the relationship between corresponding posture status judgment parameters. The signal analysis unit 208 (or the analysis unit 302), for example, can set parameters to perform or assist a (posture) state determination. For example, the parameter a can be set, and the parameter α is the sum of the differences between the posture state judgment parameters R1˜Rn of the right sensing elements and the posture state judgment parameters L1˜Ln of the left sensing elements, but the present disclosure is not limited thereto. The signal analysis unit 208 can determine the (posture) state of the bedridden person, for example, according to the relationship between the parameter α and a setting threshold Bth1, and the relationship between the parameter Rn/Ln and a setting threshold Bth2. In some embodiments, the setting threshold Bth1 and the setting threshold Bth2 are related to the type of sensor, the size of the sensor, and the body shape and weight of the user, but the present disclosure is not limited thereto. In some embodiments, the setting threshold Bth1 and the setting threshold Bth2 can be preset, for example, when the bedridden person is lying supine, but the present disclosure is not limited thereto. In some embodiments, the setting threshold Bth1 may be, for example, 0, and the setting threshold Bth2 may be, for example, 1, but the present disclosure is not limited thereto. Table 1 is a schematic table for the signal analysis unit 208 of the first sensor to determine the (posture) state of the bedridden person according to the posture state judgment parameter α.

TABLE 1

| | Relation between the posture |
|---|---|
| | state judgment parameters |
| (posture) state | and the setting threshold |
| Lying supine | α~Bth1, and Rn/Ln~Bth2 |
| Lying in the right lateral position | α > Bth1, and Rn/Ln >>Bth2 |
| Lying in the left lateral position | α < Bth1, and Rn/Ln <<Bth2 |

When the signal analysis unit 208 executes the (posture) state determination and obtains that the posture state judgment parameter α is approximately equal to the setting threshold Bth1, and the posture state judgment parameter Rn/Ln is approximately equal to the setting threshold Bth2, then the signal analysis unit 208 determines that the bedridden person is lying supine. When the signal analysis unit 208 executes the (posture) state determination and obtains that the posture state judgment parameter α is larger than the setting threshold Bth1, and the posture state judgment parameter Rn/Ln is much larger than the setting threshold Bth2, then the signal analysis unit 208 determines that the bedridden person is lying in the right lateral position. When the signal analysis unit 208 executes the (posture) state determination and obtains that the posture state judgment parameter a is less than the setting threshold Bth1, and the posture state judgment parameter Rn/Ln is much less than the setting threshold Bth2, then the signal analysis unit 208 determines that the bedridden person is lying in the left lateral position. In some embodiments, the setting threshold Bth1 can be, for example, 0, and the setting threshold Bth2 can be, for example, 1, but the present disclosure is not limited thereto.

In some embodiments, the signal analysis unit 208 can also determine whether the bedridden person has been moved correctly through the relationship between other posture state judgment parameters. For example, when the bedridden person is lying supine, for example, the average value (or sum) of the posture state judgment parameters R1˜Rn of the right sensing elements is approximately equal to the average value (or sum) of the posture state judgment parameters L1˜Ln of the left sensing elements. The ratio of the average value (or sum) of the posture state judgment parameters R1˜Rn of the side sensing elements to the average value (or sum) of the posture state judgment parameters L1˜Ln of the left sensing elements is approximately equal to Bth2, but the present disclosure is not limited thereto. That is, when the bedridden person is lying supine, the sensing signals output by the right sensing elements are approximately equal to the sensing signals output by the left sensing elements. In some embodiments, when the bedridden is lying in the right lateral position, the sensing signals output by the right sensing elements are larger than the sensing signals output by the left sensing elements. In some embodiments, when the bedridden is lying in the left lateral position, the sensing signals output by the left sensing elements are larger than the sensing signals output by the right sensing elements.

In some embodiments of FIG. 4A, the signal analysis unit 208 can obtain initial posture state judgment parameters α0, R0n, L0n according to the initial-state information, and obtain body movement posture state judgment parameters α1, R1n, L1n according to the first-state information. The signal analysis unit 208 determines whether the bedridden person has been moved correctly (turned) according to the relationship between parameter a and setting threshold Bth1, and the relationship between parameter Rn/Ln and setting threshold Bth2.

Table 2 is a schematic table for the signal analysis unit 208 of the first sensor to determine whether the bedridden person has been moved correctly according to the posture state judgment parameters. It is assured that the setting threshold Bth1 is 0, and the setting threshold Bth2 is 1.

TABLE 2

| | Relationship between |
|---|---|
| | the posture state |
| | judgment parameters and |
| Body movement state | the threshold setting |
| Supine and turning to the right | α0~0, and R0n/L0n~1 [symbol] |
| | α1 > 0, and R1n/L1n >>1 |
| Lying supine and turning to the left | α0~0, and R0n/L0n~1 [symbol] |
| | α1 < 0, and R1n/L1n <<1 |
| Lying in the right lateral position and | α0 > 0, and R0n/L0n >>1 [symbol] |
| turning to the left | α1 < 0, and R1n/L1n <<1 |
| Lying in the left lateral position and | α0 < 0, and R0n/L0n <<1 [symbol] |
| turning to the right | α1 > 0, and R1n/L1n >>1 |
| Lying in the right lateral position and | α0 > 0, and R0n/L0n >>1 [symbol] |
| turning to the up | α1~0, and R1n/L1n ~1 |
| Lying in the left lateral position and | α0 < 0, and R0n/L0n <<1 [symbol] |
| turning to the up | α1~0, and R1n/L1n ~1 |

When the signal analysis unit 208 executes (posture) state determination, it is obtained that the initial posture state judgment parameter a0 is originally approximately equal to 0, and the body movement posture state judgment parameter a1 is changed to be greater than 0. The initial posture state judgment parameter R0n/L0n is approximately equal to 1, and the body movement posture state judgment parameter R1n/L1n is changed much larger than 1. Therefore, the signal analysis unit 208 determines the movement of the bedridden person is “lying supine and turning to the right”. Similarly, when the signal analysis unit 208 executes (posture) state determination, it is obtained that the initial posture state judgment parameter α0 is originally greater than 0, and the body movement posture state judgment parameter α1 is changed to be less than 0. The initial posture state judgment parameter R0n/L0n is much larger than 1, and the body movement posture state judgment parameter R1n/L1n is changed much less than 1. Therefore, the signal analysis unit 208 determines the movement of the bedridden person is “lying in the right lateral position and turning to the left”. The determination of other posture states in Table 2 is the same as the above description, so no more details. In FIG. 4A, body movement state \textcircled{1} is lying supine and turning to the right, and body movement state \textcircled{2} is lying in the right lateral position and turning to the left.

FIG. 4B is a schematic diagram of sensing the movement sate of the bedridden person by using a second sensor in accordance with some embodiments of the present disclosure. In some embodiments, the second sensor is an image sensor. The second sensor detects the image of the bedridden person through the image sensing elements in the lens. The signal analysis unit 208 of the second sensor converts the image signals detected by the image sensing elements into corresponding state information, and generates corresponding posture state judgment parameters. The signal analysis unit 208 converts the sensing signals output by each sensing element into corresponding state information. The signal analysis unit 208 obtains the posture state judgment parameter R1′ corresponding to the first right sensing element (such as the right palm of the bedridden person), and the posture state judgment parameter R2′ corresponding to the second right sensing element (such as the right knee of the bedridden person). The signal analysis unit 208 obtains the posture state judgment parameter R3′ corresponding to the third right sensing element (such as the right footboard of the bedridden person), and the posture state judgment parameter Rn′ corresponding to the nth right sensing element. The signal analysis unit 208 obtains the posture state judgment parameter L1′ corresponding to the first left sensing element (such as the left palm of the bedridden person), and the posture state judgment parameter L2′ corresponding to the second left sensing element (such as the left knee of the bedridden person). The signal analysis unit 208 obtains the posture state judgment parameter L3′ corresponding to the third left sensing element (such as the left footboard of the bedridden person), and the posture state judgment parameter Ln′ corresponding to the nth left sensing element.

The signal analysis unit 208 determines whether the bedridden person has been moved correctly according to the relationship between corresponding posture state judgment parameters. The signal analysis unit 208 (or the analysis unit 302), for example, can set parameters to perform or assist a (posture) state determination. For example, the parameter α′ can be set. The parameter α′ is the difference between the sum of the posture judgment parameters R1′˜Rn′ of the right sensing elements and the sum of the posture judgment parameters L1′˜Ln′ of the left sensing elements. In some embodiments, for example, the concept of coordinates can be used to output the sensing position of the corresponding body part. For example, the middle of the bed is the zero point, the right side of the bed is the positive direction, and the left side of the bed is the negative direction, but the present disclosure is not limited thereto. For example, if the coordinates of the sensing element corresponding to the right side of the body of the bedridden person are located on the right side of the bed, the sensing values (such as parameters R1′˜Rn′) are positive. If the coordinates of the sensing element corresponding to the left side of the body of the bedridden person are located on the left side of the bed, the sensing values (such as parameters L1′˜Ln′) are negative, but the present disclosure is not limited thereto. The signal analysis unit 208 determines the (posture) state of the bedridden person according to the relationship between the parameter α′ and a setting threshold Bth1′, and the relationship between the parameters Rn′ and Ln′ and 0. The setting threshold Bth1′is related to the type of sensor, the size of the sensor, and the body shape and weight of the bedridden person, but the present disclosure is not limited thereto. In some embodiments, the setting threshold Bth1′ and the setting threshold Bth2′ can be preset, for example, when the bedridden person is lying supine, but the present disclosure is not limited thereto.

Table 3 is a schematic table for the signal analysis unit 208 of the second sensor to determine the (posture) state of the bedridden person according to the posture state judgment parameters.

TABLE 3

| | Relationship between the posture |
|---|---|
| | state judgment parameters and the |
| (posture) state | threshold setting |
| Lying supine | α′ > Bth1′, and Rn′ > 0 and Ln′ < 0 |
| Lying in the right lateral position | α′~Bth1′, and Rn′ > 0 and Ln′ > 0 |
| Lying in the left lateral position | α′~Bth1′, and Rn′ < 0 and Ln′ < 0 |

When the signal analysis unit 208 executes (posture) state determination, it is obtained that the posture state judgment parameter α′ is larger than the setting threshold Bth1, the posture state judgment parameter Rn′ is greater than 0, and the posture state judgment parameter Ln′ is less than 0. Therefore, the signal analysis unit 208 determines that the bedridden person is lying supine. When the signal analysis unit 208 executes (posture) state determination, it is obtained that the posture state judgment parameter α′ is approximately equal to the setting threshold Bth1′, the posture state judgment parameter Rn′ is greater than 0, and the posture state judgment parameter Ln′ is greater than 0. Therefore, the signal analysis unit 208 determines that the bedridden person is lying in the right lateral position. When the signal analysis unit 208 executes (posture) state determination, it is obtained that the posture state judgment parameter α′ is approximately equal to the setting threshold Bth1′, the posture state judgment parameter Rn′ is less than 0, and the posture state judgment parameter Ln′ is less than 0. Therefore, the signal analysis unit 208 determines that the bedridden person is lying in the left lateral position. In some embodiments, the setting threshold Bth1′ may be, for example, 0, but the present disclosure is not limited thereto. The method for the second sensor to determine whether the bedridden person has been moved correctly according to the posture state judgment parameters is the same as that of the first sensor. For example, the second sensor refers to Table 3 to determine the correct movement of the bedridden person, and the first sensor refers to Table 1 to determine the correct movement of the bedridden person, but the present disclosure is not limited thereto. In FIG. 4B, body movement state \textcircled{1} is lying supine and turning to the right, and body movement state \textcircled{2} is lying in the right lateral position and turning to the left.

FIG. 4C is a schematic diagram of sensing the movement sate of the bedridden person by using the first sensor and the second sensor in accordance with some embodiments of the present disclosure. The configuration and operation method of the sensing element included in the first sensor in FIG. 4C are the same as the configuration and operation method of the sensing element in FIG. 4A, so it will not be repeated here. The configuration and operation method of the second sensor in FIG. 4C are the same as the configuration and operation method of the second sensor in FIG. 4B, so it will not be repeated here. Specifically, the first sensor in FIG. 4C may be, for example, a pressure sensor. The second sensor in FIG. 4C may be, for example, an image sensor, but the present disclosure is not limited thereto. The image sensor can provide a more intuitive posture image and whether there is a decompression pillow placed where the hands and feet overlap. The pressure sensor can provide information on the pressure distribution position and time changes, and can adjust the turning-over strategy according to different patients and different bed rest conditions.

In some embodiments of FIG. 4C, the posture state judgment parameter R1′ is the posture state judgment parameter corresponding to the right palm after the bedridden person has been turned over. The posture state judgment parameter L1′ is the posture state judgment parameter corresponding to the left palm after the bedridden person has been turned over. Similarly, the posture state judgment parameter R2′ is the posture state judgment parameter corresponding to the right knee after the bedridden person has been turned over. The posture state judgment parameter L2′ is the posture state judgment parameter corresponding to the left knee after the bedridden person has been turned over. The posture state judgment parameter R3′ is the posture state judgment parameter corresponding to the right footboard after the bedridden person has been turned over. The posture state judgment parameter L3′ is the posture state judgment parameter corresponding to the left footboard after the bedridden person has been turned over. In FIG. 4C, two sensors are used at the same time. By comparing and combining the two images and pressure data, it can further improve the accuracy of turning/posture judgment and strategy adjustment, and improve user experience. The calculation and judgment rules of the above examples are examples, including but not limited to the methods mentioned in the examples.

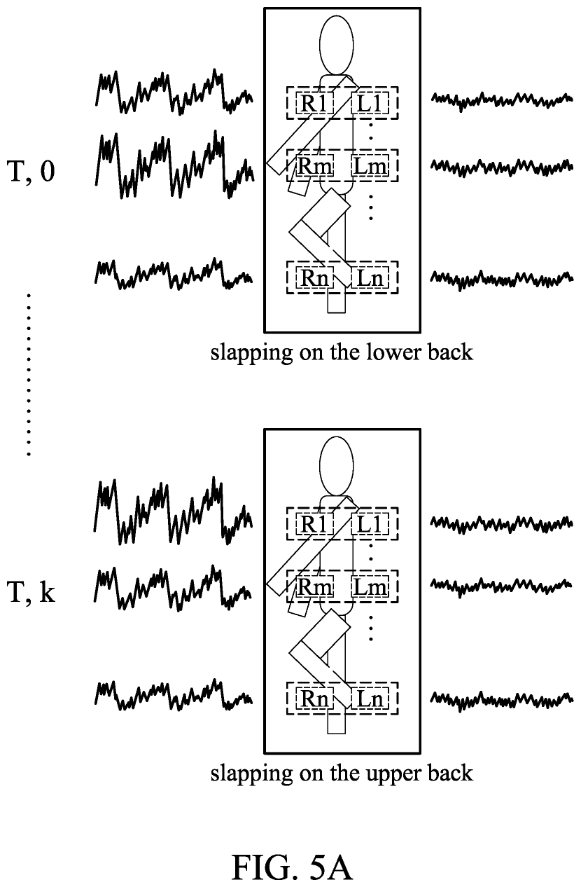

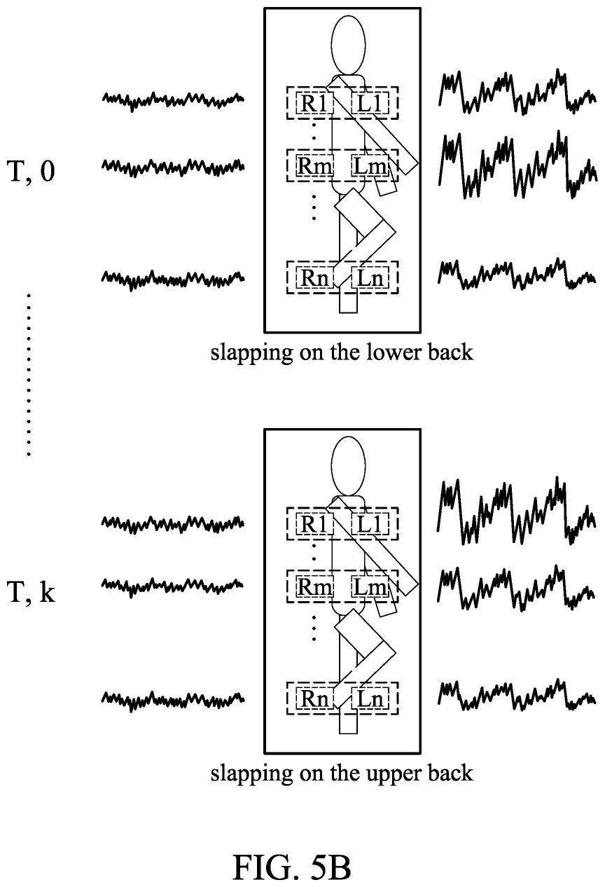

FIG. 5A is a schematic diagram of sensing by using the first sensor and slapping the back of the bedridden person in accordance with some embodiments of the present disclosure. Please refer to FIG. 5A and FIG. 4A at the same time. The signal analysis unit 208 of the first sensor can monitor the state information of the bedridden person after the movement action, to determine whether the caregiver is performing the slapping action on the bedridden person's body. As shown in FIG. 5A, in cases where the initial state of the bedridden person is lying in the right lateral position, from time point T,0 to time point T,k, the signal analysis unit 208 of the first sensor respectively receives severe signals from the first right sensing element and the mth right sensing element in the first sensor status information, and it receives minor status information from the nth right sensing element. wherein m and n are positive integers, and m is different from n. Therefore, the signal analysis unit 208 can determine that the back (e.g. the upper back and the lower back) of the bedridden person has been messaged.

Similarly, FIG. 5B is a schematic diagram of sensing by using the first sensor and slapping the back of the bedridden person in accordance with some embodiments of the present disclosure. As shown in FIG. 5B, in cases where the initial state of the bedridden person is lying in the left lateral position, from time point T,0 to time point T,k, the signal analysis unit 208 of the first sensor receives the severe state information from the first left sensing element and the mth left sensing element in the first sensor, and it receives minor status information from the nth left sensing element. Therefore, the signal analysis unit 208 can determine that the back (e.g., upper back and lower back) of the bedridden person has been slapped.

This disclosure provides a decubitus sores warning and prevention system and its monitoring method, which are used to assist caregiver by giving them regular reminders to turn the patient and determine whether the posture changes are sufficient, so as to achieve the technical effect of preventing decubitus sores.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A care system monitoring method for sensing a movement state of a bedridden person, comprising:
sensing initial-state information when the bedridden person is in an initial state;
issuing a first warning signal to remind a caregiver to perform a movement action on the bedridden person when the time that the bedridden person has been in the initial state exceeds a threshold period;
sensing first-state information when the bedridden person is in a first state after completing the movement action;
determining whether the bedridden person was moved correctly according to the initial-state information and the first-state information;
resetting the first-state information as the initial-state information if it is determined that the bedridden person has been moved correctly; and
issuing a second warning signal if it is determined that the bedridden person has been moved incorrectly.

2. The care system monitoring method as claimed in claim 1, further comprising:
continuously monitoring body-movement information when the time that the bedridden person has been in the initial state exceeds the threshold period, and determining whether the bedridden person has been moved according to the body-movement information.

3. The care system monitoring method as claimed in claim 1, further comprising:
continuously monitoring body-movement information when the time that the bedridden person has been in the initial state does not exceed the threshold period, and determining whether the bedridden person has been moved according to the body-movement information;
sensing second-state information in a second state after the bedridden person has been moved;
determining whether the bedridden person was moved correctly according to the initial-state information and the second-state information;
resetting the second-state information as the initial-state information if it is determined that the bedridden person has been moved correctly; and
issuing a third warning signal if it is determined that the bedridden person has been moved incorrectly.

4. The care system monitoring method as claimed in claim 1, wherein the form of the movement action is turning.

5. The care system monitoring method as claimed in claim 1, further comprising:
monitoring the first-state information after the movement action has been performed on the bedridden person and determining whether the caregiver is performing a slapping action on the bedridden person's body.

6. The care system monitoring method as claimed in claim 1, further comprising:
determining whether the bedridden person is lying supine, or lying in the right lateral position, or lying in the left lateral position according to the relationship between posture state judgment parameters.

7. The care system monitoring method as claimed in claim 6, wherein the relationship between the posture state judgment parameters is the sum of the differences of the posture state judgment parameters, the quotient of the posture state judgment parameters, and setting thresholds.

8. The care system monitoring method as claimed in claim 7, wherein the setting thresholds are related to the type of sensor, the size of the sensor, and the body shape and weight of the bedridden person.

9. The care system monitoring method as claimed in claim 1, further comprising:
determining whether the bedridden person has been moved correctly according to the relationship between posture state judgment parameters.

10. The care system monitoring method as claimed in claim 9, wherein the relationship between the posture state judgment parameters is the sum of the differences of the posture state judgment parameters, the quotient of the posture state judgment parameters, and setting thresholds.

11. A care system for sensing the movement state of a bedridden person, comprising:
a first sensor, configured to sense initial-state information before a movement action is performed on the bedridden person and sense first-state information after the movement action has been performed on the bedridden person;
a signal analysis unit, configured to receive the initial-state information, determine whether the time that the bedridden person has been in an initial state exceeds the threshold period, and issue a first warning signal if the time that the bedridden person has been in the initial state exceeds the threshold period; and
a warning unit, configured to receive the first warning signal and notify the caregiver to perform the movement action on the bedridden person;
wherein the signal analysis unit further receives the first-state information, and determines whether the bedridden person has been moved correctly according to the initial-state information and the first-state information after the movement action has been performed on the bedridden person;
the signal analysis unit resets the first-state information as the initial-state information if it is determined that the bedridden person has been moved correctly; the signal analysis unit issues a second warning signal if it is determined that the bedridden person has been moved incorrectly.

12. The care system as claimed in claim 11, wherein the signal analysis unit is disposed in the first sensor.

13. The care system as claimed in claim 11, wherein the signal analysis unit comprises a timing element and an analysis element; the timing element determines whether the time that the bedridden person has been in the initial state exceeds the threshold period;
the timing element issues the first warning signal if the time that the bedridden person has been in the initial state exceeds the threshold period; and
the analysis element determines whether the bedridden person was moved correctly according to the initial-state information and the first-state information.

14. The care system as claimed in claim 11, further comprising:
a data storage, configured to receive and store the initial-state information and the first-state information.

15. The care system as claimed in claim 14, wherein the signal analysis unit is disposed in the data storage.

16. The care system as claimed in claim 11, wherein the first sensor comprises at least two sensing elements corresponding to different body parts of the bedridden person.

17. The care system as claimed in claim 11, further comprising:
a second sensor, configured to sense the movement action of the bedridden person;
wherein the second sensor is different from the first sensor.

18. The care system as claimed in claim 16, wherein the at least two sensing elements provide sensing signals corresponding to different body parts of the bedridden person.

19. The care system as claimed in claim 18, wherein the first sensor comprises a signal processing unit; the signal processing unit performs analog-to-digital conversion, filtering, and amplification on the sensing signals from the at least two sensing elements to generate the initial-state information and the first-state information after the movement action.

20. The care system as claimed in claim 16, wherein the signal analysis unit determines whether the bedridden person has been moved correctly according to the relationship between posture state judgment parameters corresponding to the at least two sensing elements.
